(19) United States
(12) Patent Application Publication
(10) Pub. No.: US 2025/0019607 A1
(43) Pub. Date: Jan. 16, 2025

(54) METHODS FOR THERMAL OLIGOMERIZATION TO LIQUID FUEL RANGE PRODUCTS

(71) Applicant: PURDUE RESEARCH FOUNDATION, WEST LAFAYETTE, IN (US)

(72) Inventors: Jeffrey T. Miller, Naperville, IL (US); Matthew A. Conrad, El Segundo, CA (US); Jaiden Deline, West Lafayette, IN (US)

(21) Appl. No.: 18/706,745

(22) PCT Filed: Nov. 4, 2022
(86) PCT No.: PCT/US2022/049008

§ 371 Date: May 2, 2024

Related U.S. Application Data
(60) Provisional application No. 63/276,275, filed on Nov. 5, 2021.

(51) Int. Cl. C10L 1/04; C07C 2/04; C10G 50/00

(52) U.S. Cl. CPC C10L 1/04; C07C 2/04; C10G 50/00; C10G 2300/1088

(57) ABSTRACT

Methods for making light hydrocarbon oligomers and the oligomer products made therefrom. One or more C₂ to C₁₂ olefins are reacted with at least one porous support material at a temperature of about 200° C. to 500° C. to provide a higher molecular weight product comprising C₃ to C₂₆ olefins. The at least one porous support material is not a traditional catalyst and contains no additional catalytic metals, activators, promoters or acid sites.

### CROSS-REFERENCE TO RELATED APPLICATIONS

This application claims priority to U.S. Provisional Patent Application No. 63/276,275, which was filed on Nov. 5, 2021, and which is hereby incorporated by reference in its entirety.

### GOVERNMENT RIGHTS

This invention was made with government support under EEC-1647722 awarded by the National Science Foundation. The government has certain rights in the invention.

### BACKGROUND

### Technical Field

Embodiments of the present disclosure generally relate to light hydrocarbon alkene oligomerization. More particularly, embodiments relate to catalyst development for light hydrocarbon alkene oligomerization.

### Description of the Related Art

In the past, liquid transportation fuels have been produced by catalytic cracking and reforming of large hydrocarbons in crude oil, or by C—C bond formation either from syngas in Sasol's Fischer-Tropsch plants or the oligomerization of light olefins such as ethylene, propylene, and butenes. While these processes are still in use today, due to the recent shale gas boom there is an opportunity to convert the ethane and propane found in shale gas into higher molecular weight olefins by a two-step process: (1) alkane dehydrogenation and (2) olefin oligomerization. Therefore, olefin oligomerization is an important process in potentially upgrading shale gas to higher molecular weight olefins in the C₆-C₁₆ range.

The first olefin oligomerization catalyst was phosphoric acid, discovered by Ipatieff in the 1930's, first as liquid phosphoric acid, and eventually as solid phosphoric acid (SPA) supported on Kieselguhr. SPA was quickly commercialized by UOP as the CatPoly process which operates primarily on C₃═/C₄═ feedstock at 230° C. and 13-14 bar to produce gasoline with an octane rating of 80-83 and a lifetime of several months. Liquid fuel with an octane rating of 95-100 can be obtained by selectively oligomerizing the isobutene produced followed by hydrogenation. In the 1970's, Mobil commercialized a zeolite ZSM-5 catalyst in its Mobil Olefins to Gasoline and Distillate (MOGD) oligomerization process, which converts light olefins such as ethylene, propylene, and butene into high-octane gasoline and distillates with a lifetime of 2-3 weeks. MOGD process conditions and zeolite properties can be chosen to favor lower or higher boiling products and degree of branching but is generally operated between 200 and 340° C. and 5-50 bar.

Other acidic materials such as sulfonic resins have been applied; however, SPA and zeolites are the most common. Brønsted acid catalysts operate by a carbenium ion intermediate, which gives rise to branching and conjunct polymerization, characterized by cyclization and hydrogen transfer forming isoparaffins and aromatics. Of the olefins, isobutene tends to react the fastest with acids because it forms a tertiary carbenium intermediate, whereas ethylene forms a primary carbenium ion, making it the most difficult (least reactive) olefin to oligomerize. As a result, commercial ethylene oligomerization often requires high activity bomogencous Ni²⁺ or Ti⁴⁺ catalysts despite many studies in heterogeneous systems.

The “nickel-effect,” i.e., the high affinity for Ni(II) species to catalyze olefin oligomerization compared to other transition metals, was first discovered in 1952 by Karl Ziegler, who was studying alkylaluminum ethylene polymerization when a routine polymerization experiment resulted in a high 1-butene yield. The autoclave in the laboratory was determined to have been contaminated with nickel which was highly active and selective to ethylene dimerization. The first nickel-catalyzed commercial propylene dimerization process, Dimersol, was developed by IFP in the late 1970's. Dimersol utilized a bomogencous Ni(II) complex with aluminum alkyl activators and operated near 50° C.

Upon the development of appropriate phosphorus-oxygen ligands, nickel catalysts were soon commercialized in the Shell Higher Olefins Process (SHOP) to selectively produce linear alpha-olefins with a geometric distribution of products between C₄ and C₂₀. The SHOP catalyst is a homogenous Ni(II) complex which operates between 80-120° C. and 70-140 bar in a polar solvent. An appropriate nickel ligand was also discovered that enabled ethylene to be polymerized instead of oligomerized.

Along with Dimersol and SHOP, another commercial ethylene oligomerization process is Alphabutol by IFP. Alphabutol uses a Ti(IV) complex to dimerize ethylene with high selectivity to 1-butene. The operating conditions for Alphabutol are around 55° C. and 22 bar, which yield >90 wt % butenes, with the butene distribution almost completely selective to 1-butene (99.8 wt %). A few other notable homogeneous systems involve the use of chromium complexes with Al-ethyl activators (Phillips trimerization catalyst), alkyl-aluminum complexes (developed by Gulf (now Chevron Phillips) and BP Amoco (Ethyl process)), and zirconium halides (Idemitsu Petro. Comp. Ltd), but they are not as widely used.

While homogeneous Ni(II) complexes are highly active and can be somewhat tuned in terms of their product distribution, it is economically attractive to develop heterogeneous catalysts, which do not require activators or solvents, are readily separated from the products, can often be used at higher temperatures and can be regenerated. Supported heterogeneous Ni²⁺ catalysts display high activity; however, the product distribution is mostly C₄=olefins (˜80 mol %), which are too volatile for liquid fuels. Additionally, at higher temperatures in reducing atmosphere, nickel ions reduce to inactive metallic Ni nanoparticles. The best success of nickel-catalyzed ethylene oligomerization to liquid fuel range products involves Ni²⁺ ions in zeolites or SiO₂/Al₂O₃ in which it is generally thought that acid sites in the support contribute to the activity and product distribution. OCTOL by Hüls and UOP uses a heterogenous Ni/SiO₂—Al₂O₃ catalyst for butene dimerization and is currently the only commercial heterogeneous nickel oligomerization technology. Nickel catalysts operate via the Cossee-Arlman mechanism, which consists of Ni—H and Ni—R intermediates, and olefin insertion and beta-hydride elimination elementary steps.

Nickel zeolite catalysts have demonstrated the ability to produce diesel range fuels but deactivate quickly above about 250° C. due to reduction of the nickel ions as well as pore blocking by high molecular weight products.

A third pathway for ethylene oligomerization involves the thermal radical reaction of ethylene in the gas phase. The earliest reports of thermal ethylene reactions were pre-1900 and were conducted above 500° C. The lack of analytical techniques and general understanding of chemical principles heavily limited their research. Various studies were conducted between 1900 to 1960, yet many of these still lacked the ability to obtain accurate rate and product analyses. Ipatieff was the first to report the oligomerization of ethylene at super atmospheric pressure (70 atm) from 325-400° C. Liquid products including pentenes, hexenes, and larger hydrocarbons with 21% boiling above 280° C. were reported, likely corresponding to products up to C₁₅ or C₁₆. Methods for product identification included reacting the products with sulfuric acid and bromine to determine the presence of olefins as well as combustion tests to determine the ratios of C and H in each boiling fraction, which is separated by distillation.

In 1931, Pease determined the reaction order of ethylene “polymerization” from 2.5 to 10 atm to be 2nd order with an activation energy of 146 KJ/mol from 350-500° C., with ethylene conversions ranging from 10-60%. No specific products besides hydrogen and generic linear olefins were identified in Pease's study, and he concluded based on the reaction order that 1-butene must be one of the main products. Silcocks studied this reaction in 1956 and determined an activation energy of 151 KJ/mol from data at 450 and 600° C. at atmospheric pressure. The products identified were methane, hydrogen, propylene, butenes, and “polymer.”

### SUMMARY

Methods for making light hydrocarbon oligomers and the oligomers made therefrom are provided herein. In one embodiment, one or more C₂ to C₁₂ olefins can be reacted with at least one porous support material at a temperature of about 200° C. to 500° C. to provide a higher molecular weight product comprising C₃ to C₂₆ olefins. The at least one porous support material is not a traditional catalyst and contains no additional catalytic metals, activators, promoters or acid sites.

In another embodiment, the method for making light hydrocarbon oligomers consists of reacting one or more C₂ to C₁₂ olefins with at least one porous support material at a temperature of about 200° C. to 500° C. to provide a higher molecular weight product comprising C₃ to C₂₆ olefins, wherein the at least one porous support material contains no additional catalytic metals, activators, promoters or acid sites.

A light hydrocarbon oligomer is also provided herein. The oligomer can be made from any of the foregoing methods. For instance, one or more C₂ to C₁₂ olefins can be reacted with at least one porous support material at a temperature of about 200° C. to 500° C. to provide a higher molecular weight product comprising C₃ to C₂₆ olefins, wherein the at least one porous support material contains no additional catalytic metals, activators, promoters, or acid sites. The higher molecular weight product can be a mixture of linear olefins having odd and even carbon numbers.

### BRIEF DESCRIPTION OF THE DRAWINGS

So that the manner in which the above recited features of the present disclosure can be understood in detail, a more particular description of the present disclosure may be had by reference to embodiments, some of which are illustrated in the appended drawings. It is to be noted, however, that the appended drawings illustrate only typical embodiments of the present disclosure and are, therefore, not to be considered limiting of its scope, for the disclosure may admit to other equally effective embodiments. It is emphasized that the figures are not necessarily to scale and certain features and certain views of the figures can be shown exaggerated in scale or in schematic for clarity and/or conciseness.

FIG. 1 is a Reaction Order Plot for the Empty Reactor (30 cm³), 450° C., 1 to 18 bar C₂H₄, conversion <10%.

FIG. 2 is an Arrhenius Plot for the Empty Reactor (12 cm³), 1.5 bar C₂H₄, 340-500° C., conversion <2%.

FIG. 3 is an Arrhenius Plot for SiO₂, 1.5 bar C₂H₄, 320-360° C., conversion <2% compared to the empty reactor.

FIG. 4 is an Arrhenius Plot for Al₂O₃, 1.5 bar C₂H₄, 320-360° C. and 410-470° C. compared to the empty reactor.

FIG. 5 is an Arrhenius Plot for Na-BEA, 1.5 bar C₂H₄. 320-380° C., conversion <5% compared to the empty reactor.

FIG. 6 is an Arrhenius Plot for Na—Y, 1.5 bar C₂H₄, 320-380° C., conversion <5% compared to the empty reactor.

FIGS. 7A and 7B are Arrhenius plots depicting the kinetics of ethylene reactions with different high surface area supports at temperatures ranging from 300° C. to 410° C. at (FIG. 7A) 1.5 bar and (FIG. 7B) 42-43.5 bar. The thermal reactions of ethylene from 300° C. to 410° C. are shown in open squares. Alumina is shown in triangles and silicon dioxide in circles.

FIG. 8 shows the product selectivity of ethylene in the presence of silicon dioxide compared to the thermal reaction at 360° C. and 43.0-43.5 bar. The conversions thermally and with silicon dioxide were 0.58 and 0.96%, respectively. The products are based on carbon number distribution.

FIG. 9 shows the product selectivity of ethylene in the presence of alumina compared to the thermal reaction at 385° C. and 27.5 bar. The conversions thermally and with alumina were 11.9 and 16.9%, respectively. The products are based on carbon number distribution.

FIGS. 10A and 10B show the product selectivity of ethylene with alumina at 23 bar, 360° C., and from 1-70% conversion. FIG. 10A shows the major products selectivity, and FIG. 10B shows the C₄ isomer distribution.

FIG. 11 shows the product selectivity of ethylene with alumina versus the thermal reaction at 1.5 bar, 400° C. and 0.1% conversion.

FIG. 12 shows the product selectivity of ethylene with alumina versus conversion below 20% at 1.5 bar and 360° C.

FIGS. 13A-13D show the selectivity of major products with alumina at 300 vs 360° C. at 23 bar of C₂H₄ below 20% conversion. FIG. 13A shows the shows selectivity of ethane. FIG. 13B shows the shows product selectivity of propane. FIG. 13C shows the shows product selectivity of butane. FIG. 13D shows the shows product selectivity of pentane and heavier.

FIG. 14 shows the product selectivity of ethylene with alumina versus temperatures of 300° C. and 360° C. at 7.7-8.0% conversion and 1.5 bar.

FIGS. 15A-15D show the selectivity of major products with alumina at 1.5 vs 23 bar of C₂H₄ at 360° C. below 20% conversion. FIG. 15A shows the shows selectivity of ethane.

FIG. 15B shows the shows product selectivity of propane. FIG. 15C shows the shows product selectivity of butane. FIG. 15D shows the shows product selectivity of pentane and heavier.

FIG. 16 shows the product selectivity of ethylene with alumina at 1.5 vs 43.0 bar and 360° C. at 7.3-7.7% conversion.

FIG. 17 is an Arrhenius plot depicting the kinetics of propylene reactions with alumina from 260 to 300° C. at 1.5 bar, X<10%. The thermal reactions of propylene from 400 to 500° C. are shown in open triangles for comparison.

FIG. 18 shows the product selectivity of propylene with alumina at 2.0% conversion at 260° C. and 1.5 bar.

FIG. 19 shows the product selectivity of propylene with alumina versus temperatures from 260 to 300° C. at 12.1-14.4% conversion at 1.5 bar.

FIG. 20 shows the product selectivity of propylene with alumina versus conversion from 7.4 to 25.2% at 300° C. and 1.5 bar.

### DETAILED DESCRIPTION

It is to be understood that the following disclosure describes several exemplary embodiments for implementing different features, structures, or functions. Exemplary embodiments of components, arrangements, and configurations are described below to simplify the present disclosure; however, these embodiments are provided merely as examples and are not intended to limit the scope of the claimed invention. Additionally, the present disclosure can repeat reference numerals and/or letters in the various embodiments and across the figures provided herein. This repetition is for the purpose of simplicity and clarity and does not in itself dictate a relationship between the various embodiments and/or configurations. Moreover, the exemplary embodiments presented below can be combined in any combination of ways, i.e., any element from one exemplary embodiment can be used in any other exemplary embodiment, without departing from the scope of the disclosure.

Additionally, certain terms are used throughout the following description and claims to refer to particular components. As one skilled in the art will appreciate, various entities can refer to the same component by different names, and as such, the naming convention for the elements described herein is not intended to limit the scope of the claimed invention, unless otherwise specifically defined herein. Further, the naming convention used herein is not intended to distinguish between components that differ in name but not function.

Furthermore, in the following discussion and in the claims, the terms “including” and “comprising” are used in an open-ended fashion, and thus should be interpreted to mean “including, but not limited to.” The phrase “consisting essentially of” means that the described/claimed composition does not include any other components that will materially alter its properties by any more than 5% of that property, and in any case does not include any other component to a level greater than 3 mass %.

Unless otherwise indicated, all numerical values are “about” or “approximately” the indicated value, meaning the values take into account experimental error, machine tolerances and other variations that would be expected by a person having ordinary skill in the art. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for making the measurement.

The term “or” is intended to encompass both exclusive and inclusive cases, i.e., “A or B” is intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins are used, unless specified to the contrary or the context clearly indicates that only one olefin is used.

The terms “alkane” and “paraffin” are used interchangeably, and both refer to any saturated molecule containing hydrogen and carbon atoms only, in which all the carbon-carbon bonds are single bonds and are saturated with hydrogen. Such saturated molecules can be linear, branched, and/or cyclic.

The terms “alkene” and “olefin” are used interchangeably, and both refer to any unsaturated molecule containing hydrogen and carbon atoms only, in which one or more pairs of carbon atoms are linked by a double bond. Such unsaturated molecules can be linear, branched, or cyclic, and can include one, two, three or more pairs of carbon atoms linked by double bounds (i.e. mono-olefins, di-olefins, tri-olefins, etc).

The term “wt %” means percentage by weight, “vol %” means percentage by volume, “mol %” means percentage by mole, “ppm” means parts per million, and “ppm wt” and “ppmw” are used interchangeably and mean parts per million on a weight basis. All concentrations herein, unless otherwise stated, are expressed on the basis of the total amount of the composition in question.

Each of the appended claims defines a separate invention, which for infringement purposes is recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims.

Each of the inventions will now be described in greater detail below, including specific embodiments, versions and examples, but the inventions are not limited to these embodiments, versions or examples, which are included to enable a person having ordinary skill in the art to make and use the inventions, when the information in this disclosure is combined with publicly available information and technology.

Methods for enhancing thermal ethylene oligomerization via addition of one or more high surface area supports, which mediate the activation of ethylene, resulting in a significant rate increase in the production of higher molecular weight, fuel-range hydrocarbons, are provided. While thermal oligomerization of olefins has been studied previously, it is not commercialized today in part due to the low thermal reaction rates of olefins at low concentrations. Unlike previous catalytic materials for olefin activation and conversion, the one or more high surface area supports provided herein can physically adsorb olefins, which activates the olefins for thermally induced reactions. The high surface area oxides also increase the yield of higher molecular weight products to provide higher selectivity to products with more than C₄ olefins having even and/or odd number of carbon atoms.

It has been surprisingly and unexpectedly discovered that thermally initiated olefin, and especially ethylene, oligomerization at 200° C. or higher at pressures ranging from 1 bar-45 bar is enhanced by the addition of one or more high surface area supports provided herein. The gas phase reaction results in little methane or coking (i.e., <2 mol %, or <1 mol %, or <0.5 mol %) and can run for at least a week without significant loss of conversion. The main products are liquid fuel range oligomers that can be blended into gasoline or hydrogenated to produce premium diesel fuel. The main products can include even and odd carbon products ranging from propylene up to about C₁₀ olefins. For example, the main products for ethylene oligomerization can include C₃H₆ and C₄H₈ oligomers as well as small amounts of CH₄ and C₂H₆, due to secondary olefin reactions. At high pressures and conversions, the products contain C₂-C₁₈ olefins.

Even more surprisingly, it has been discovered that olefin products of all carbon numbers result, independent of the reactant olefin. The high porous support materials can be used to obtain different product distributions by varying any one or more of temperature, pressure, and ethylene conversion. Indeed, it has been surprisingly and unexpectedly discovered that the high porous support materials provide an unexpected distribution of all carbon numbers. For example, oligomerization of ethylene would be expected to give even carbon numbered products; however, olefins with 3, 5, 7, 9, etc., carbons can be produced. For ethylene, the products obtained can have 3, 4, 5, 6, 7, or more carbons, which is nothing short of surprising and unique. These products make excellent hydrocarbons for gasoline and diesel fuels.

In certain embodiments, the amount of each olefin with an odd number of carbons (e.g., 3, 5, 7, 9, etc.) resulting from the oligomerization of ethylene can range from a low of about 0.1 mol %, 0.5 mol %, or 1 mol % to a high of about 3 mol %, 5 mol %, or 10 mol %. Each olefin with an odd number of carbons (e.g., 3, 5, 7, 9, etc.) resulting from the oligomerization of ethylene can also be at least 0.1 mol %, at least 0.5 mol %, at least 1 mol %, at least 2 mol %, at least 3 mol %, at least 5 mol %, or at least 10 mol %.

By “oligomer(s)”, it is meant dimers, trimers, tetramers, and other molecular complexes having less than 26 repeating units. Additionally, olefin products with carbon numbers, which are not multiples of the starting reactant are also produced. Oligomers provided herein are typically gases or liquids at ambient temperature, and can include low melting solids, including waxes, at ambient temperature. In some embodiments, the oligomers provided herein, which are suitable for production of gasoline and diesel fuels, can have an atomic weight or molecular weight of less than 1000 AMU (Da), such as about 500 or less, 400 or less, 300 or less, or 200 or less. The molecular weight of the oligomer, for example, can range from a low of about 50, 250 or 350 to a high of about 500, or 1,000 AMU (Da).

Considering the high surface area porous support material or carrier in more detail, the support material does not require co-catalysts, modification, promoters, impregnation, or any type of activation. Said another way, the support material contains no added catalytic metals or oxides, activators, promoters, or acid sites. The high surface area support material does not function like a catalyst. A typical catalyst activates the reactants and controls the reaction products. The high surface area support materials do not activate the olefins but can modify the thermally driven olefin reactions and can modify the products that are formed and how fast the reaction occurs.

The high surface area, porous support material or carrier can be, or can include, metal oxides, mixed oxides, inorganic oxides, zeolites, carbon and other high temperature stable (up to about 500° C.) microporous and mesoporous supports. The high surface area porous support material or carrier can further include silica, which may or may not be dehydrated, fumed silica, alumina, silica-alumina, or mixtures thereof. Other suitable support materials can include magnesia, titania, zirconia, montmorillonite, phyllosilicate, clays, and the like. Suitable support materials can also include nanocomposites and aerogels. In certain embodiments, the high surface area porous support material is or includes any one or more of the following: silicon dioxide (SiO₂), aluminum oxide (Al₂O₃), titanium dioxide, silica-alumina, cerium dioxide, zirconium dioxide, magnesium oxide, silica pillared clays, metal modified silica, metal oxide modified silica, metal oxide modified silica-pillared clays, silica-pillared micas, metal oxide modified silica-pillared micas, silica-pillared tetrasilicic mica, silica-pillared tainiolite, and combinations thereof.

In certain embodiments, the high porous support material is or includes one or more zeolites. Suitable zeolites can be or can include ZSM-5, BEA, MOR, Y, AIPO-5, and other 10 and 12 ring medium and large pore size zeolites and microporous oxides. Non-acidic zeolites are preferred. As used herein, the term “non-acidic zeolite” refers to a zeolite structure having no active acid sites, i.e., H+ sites. In the non-acidic zeolites, the framework Al ions are charged balanced by alkali ions, alkaline earth ions, and rare earth ions. Suitable alkali ions can include Li, Na, K, and other group 1A elements, with an alkali to aluminum molar ratio of 1.0 or higher. Similarly, alkaline earth ions, such as Mg, Ca, Sr and other group 2A metals with a +2 charge, can ion exchange Al ions with a 1:2 molar ratio; while rare earth ions, for example, La, Ce, and other lanthanide ions with a charge of +3, can charge balance 3 aluminum ions in the zeolite. Mixtures of alkali ions, alkaline earth ions, and rare earth ions with a cation charge to aluminum ratio of 1.0 or higher are also suitable.

Combinations of any two or more high porous support materials or carriers can be used. The foregoing supports are commercially available or can be prepared using techniques known to those skilled in the catalysis art.

In one or more preferred embodiments, the high porous support material or carrier is Al₂O₃ or SiO₂ or a combination thereof. In one or more preferred embodiments, the high porous support material or carrier is Na-BEA, Na—Y, or a combination thereof. In one or more other preferred embodiments, the high porous support material or carrier is Al₂O₃, SiO₂, Na-BEA, Na—Y, or combinations thereof.

A suitable porous support material can have a surface area in the range of from about 10 m²/g to about 700 m²/g, a pore volume in the range of from about 0.1 cc/g to about 4.0 cc/g, and an average particle size in the range of from about 5 μm to about 500 μm. More preferably, the porous support material can have a surface area in the range of from about 50 m²/g to about 500 m²/g, a pore volume in the range of from about 0.5 cc/g to about 3.5 cc/g, and an average particle size in the range of from about 10 μm to about 200 μm. The surface area can range from a low of about 50 m²/g, 150 m²/g, or 300 m²/g to a high of about 500 m²/g. 700 m²/g, or 900 m²/g. The surface area also can range from a low of about 200 m²/g, 300 m²/g, or 400 m²/g to a high of about 600 m²/g. 800 m²/g, or 1,000 m²/g. The average pore size of the porous support material can range of from about 10 Å to 1000 Å, about 50 Å to about 500 Å, about 75 Å to about 350 Å, about 50 Å to about 300 Å, or about 75 Å to about 120 Å.

The porous support material can convert light hydrocarbon alkenes to higher molecular weight oligomers at temperatures of about 200° C. to 700° C. and pressures of 1 bar to 100 bar. Conversion of light hydrocarbon alkenes to higher molecular weight oligomers can also occur at temperatures ranging from a low of about 200° C., 250° C., 300° C., or 350° C. to a high of about 400° C., 500° C., 600° C., or 700° C. Conversion temperatures can also be about 700° C. or less. 650° C. or less, 600° C. or less, 560° C. or less, 510° C. or less, 480° C. or less, 430° C. or less, or 390° C. or less. Conversion of light hydrocarbon alkenes to higher molecular weight oligomers can occur at pressures ranging from a low of about 1 bar, 10 bar, 20 bar, or 30 bar to a high of about 50 bar, 60 bar, 80 bar, or 100 bar. The pressure can also be about 1 bar or less, 10 bar or less, 20 bar or less, 30 bar or less, 40 bar or less, 50 bar, or 75 bar or less.

The light hydrocarbons or hydrocarbon feed stream can be or can include natural gas, natural gas liquids, or mixtures of both. The hydrocarbon feed stream can be derived directly from shale gas or other formations. The hydrocarbon feed stream can also originate from a refinery, such as from a fluid catalytic cracking (FCC) unit, coker, or steam cracker, and pyrolysis gasoline (pygas) as well as alkane dehydrogenation processes, for example, ethane, propane and butane dehydrogenation.

The hydrocarbon feed stream can be or can include one or more olefins having from about 2 to about 12 carbon atoms (such as about 2 to 12 carbon atoms, 2 to about 12 carbon atoms, or 2 to 12 carbon atoms). The hydrocarbon feed stream can be or can include one or more linear alpha olefins, such as ethene, a propene, a butene, a pentene and/or a hexene. The process is especially applicable to ethene and propene oligomerization for making C₄ to about C₂₆ oligomers.

The hydrocarbon feed stream can contain greater than about 65 wt % olefins, such as greater than about 70 wt. % olefins or greater than about 75 wt % olefins. For example, the hydrocarbon feed stream can contain one or more C₂ to C₁₂ olefins in amounts ranging from a low of about 50 wt %. 60 wt % or 65 wt % to a high of about 70 wt %. 85 wt % or 100 wt %, based on the total weight of the feed stream. The hydrocarbon feed stream also can include up to 80 mol % alkanes, for example, methane, ethane, propane, butane, and/or pentane; although the alkane generally comprises less than about 50 mol % of the hydrocarbon feed stream, and preferably less than about 20 mol % of the hydrocarbon stream.

The hydrocarbon feed can have a temperature of 200° C. or higher. For example, the temperature of the hydrocarbon feed can range from a low of about 200° C., 300° C., or 350° C. to as high of about 500° C., 600° C., or 700° C. The temperature of the hydrocarbon feed also can be 200° C. or less, 250° C. or less, 300° C. or less, 350° C. or less, 380° C. or less, 400° C. or less, 425° C. or less, 450° C. or less, 460° C. or less, 470° C. or less, or 475° C. or less, or 500° C. or less.

The resulting oligomer(s) can be or can include one or more olefins having from 4 to 26 carbon atoms, such as 12 to 20 carbon atoms, or 16 to 20 carbon atoms. The resulting oligomers, for example, can include butene, hexene, octene, decene, dodecene, tetradecane, hexadecane, octadecene and eicosene and higher olefins, as well as any combinations thereof. The resulting oligomer(s) also can have less than about 5% aromatics (such as less than 5%, less than 4%, less than 3%, less than 2% or less than 1%) and less than about 10 ppm sulfur (such as less than 10 ppm, less than 7.5 ppm, less than 5 ppm, or less than 2.5 ppm). The resulting oligomer(s) also can have zero or substantially no aromatics and zero or substantially no sulfur.

The resulting oligomer(s) can be useful as precursors, feedstocks, monomers and/or comonomers for various commercial and industrial uses including polymers, plastics, rubbers, elastomers, as well as chemicals. For example, these resulting oligomer(s) are also useful for making polybutene-1, polyethylene, polypropylene, polyalpha olefins, block copolymers, detergents, alcohols, surfactants, oilfield chemicals, solvents, lubricants, plasticizers, alkyl amines, alkyl succinic anhydrides, waxes, and many other specialty chemicals.

The resulting oligomer(s) can be especially useful for production of diesel and jet fuels, or as a fuel additive. In certain embodiments, the resulting oligomer(s) can have a boiling point in the range of 170° C. to 360° C. and more particularly 200° C. to 300° C. The resulting oligomer(s) also can have a Cetane Index (CI) of 30 to 100 and more particularly 40 to 60. The resulting oligomer(s) also can have a pour point of −50° C. or −40° C.

### EXAMPLES

The following examples serve to illustrate the present disclosure. The examples are not intended to limit the scope of the claimed invention in any way. The effects of a high surface area support such as SiO₂, Al₂O₃, or a non-acidic zeolite were studied in comparison to an empty reactor (i.e., a reactor without any added support). Higher ethylene conversion rates and differences in product distributions were surprisingly discovered when a high surface area support was used.

In each example, a quartz tube (10.5 mm ID, 1.1 mm thickness) approximately 14″ in total length was loaded into a clamshell furnace with insulation tape enclosing a 5″ length active reaction zone. A thermal-well placed down the axial length of the tube allowed the temperature profile to be measured at 1″ intervals. A length-averaged temperature was then calculated for each temperature setpoint. The reactor was then heated to the desired setpoint and olefin flow rate. For each data point, the product gas flow rate was verified using a bubble film flowmeter. Ultra-high purity ethylene or propylene was purchased from Indiana Oxygen and used in all experiments.

Products were analyzed using a Hewlett Packard 6890 Series Gas Chromatograph with an Agilent HP-Al/S column (25 m in length, 0.32 mm ID, and 8 μm film thickness) and flame ionization detector. To detect higher molecular weight products up to C₈, the reactor discharge lines were traced with heat tape and set to 150° C. during the experiment. The conversion and product distribution were calculated on a molar basis, assuming a closed carbon balance since no significant carbon deposition was observed over the course of experiments. A mass flow controller calibration curve was made, and so comparison of the product flow rate to the flow rate with no conversion (bypass) could be used to estimate the conversion. For tests at low conversion (X<2%), the product flow rate did not deviate from the expected flow rate, and thus a 100% carbon balance was assumed in the calculations.

To understand the product distributions and rates at higher olefin concentrations and conversions, ethylene was tested at pressures up to 43.5 bar. A 316 stainless steel tube (⅜″ ID, ⅛″ thickness) 2 feet in length, with VCR fittings at the inlet and outlet to seal the system was used. The insulation allowed a thermal reaction zone of about 16.5″ which corresponded to a volume of about 30 cm³. A thermal-well placed down the length of the tube allowed the temperature profile to be measured at 2″ intervals. A length-averaged temperature was then calculated for each temperature setpoint. The reactor was first pressurized and then the catalyst bed was heated to the desired setpoint temperature in flowing N2 and allowed to stabilize for 1 hr. Pure C₂H₄ was then flowed through the reactor. To study the effects of the thermal reaction on several different supports, the thermal reaction zone was filled with support and tested as described above. Additionally, the supports were treated for 6-8 hours for any transient effects to disappear, and the conversion was steady for multiple GC injections.

Products were analyzed using a Hewlett Packard 6890 Series Gas Chromatograph with an Agilent HP-1 column (25 m in length, 0.32 mm ID, and 8 μm film thickness) and flame ionization detector. To detect higher molecular weight products up to C₁₀, the reactor discharge lines were traced with heat tape and set to 175° C. during the experiment. The conversion and product distribution were calculated on a molar basis except at high conversions (X>20%), in which the mass selectivity was more convenient to calculate due to the condensation of considerable amounts of liquid products, which were collected in a glass vial maintained in an ice bath. Comparison of the liquid production rate and unreacted ethylene in the gas effluent to the feed mass flow rate enabled an estimation of the conversion. For lower conversions (X<20%), the product did not condense significantly. Additionally, little carbon deposition was observed over several days of testing, thus, a 100% carbon balance was assumed in the calculations.

A mass flow controller calibration curve was made, and so comparison of the product flow rate to the flow rate with no conversion served to estimate the conversion. For differential conditions (X<10%), the product flow rate did not deviate from the expected flow rate, and thus a 100% carbon balance was assumed in the calculations. For X>10%, the molar volumes of each carbon number group were obtained from Yaw's Handbook (Knovel Database) and the C₂H₄ conversion was estimated from the product volume flow rate. Products above C₅ were assumed to be saturated in the product stream at room temperature and atmospheric pressure where the bubble flowmeter was operated.

### Example 1: Empty Reactor (i.e., No Added Support)

To demonstrate the rate enhancement of a support, first the empty reactor was studied at a range of temperatures and pressures, and the kinetics were measured at low conversions (X<2%). The results of various conditions of thermal ethylene oligomerization are shown in the following tables (Tables 1-2). The data in the empty reactor demonstrate that high rates can be achieved with a pure ethylene feed at 465° C. and 31.5 bar, with a rate of 6.2×10⁶ mol C₂H₄/s/cm³. The products consist of C₃-C₁₀ olefins with less than 4% total selectivity to methane and ethane. Furthermore, little coking of the reactor was observed during the experiment which took place over about 5 days.

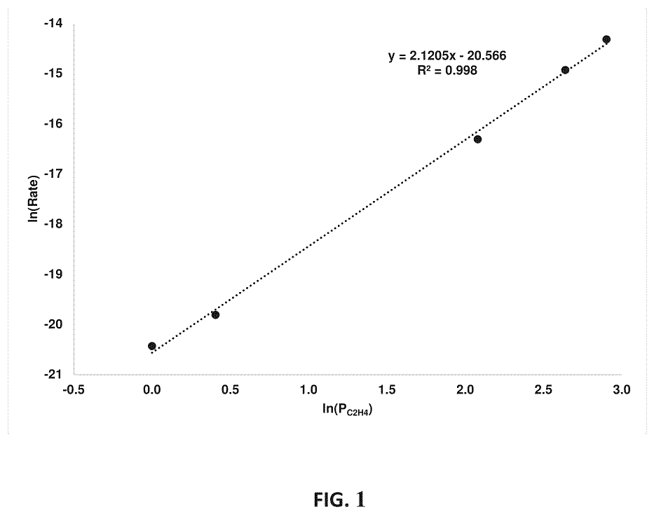

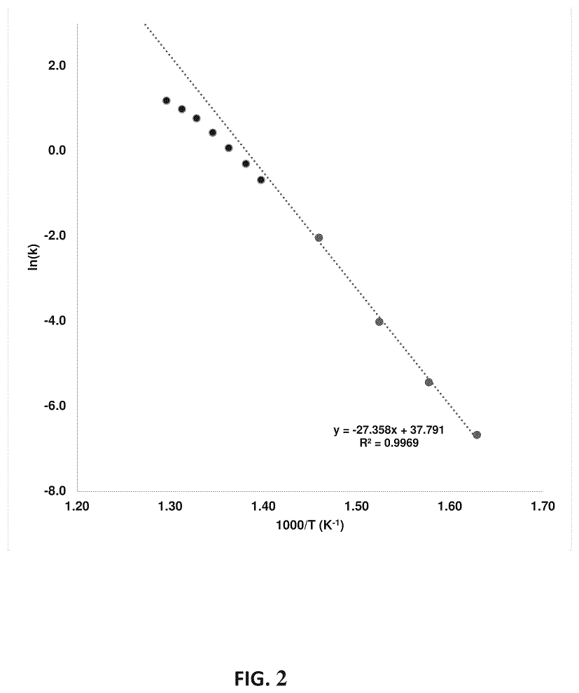

The reaction kinetics in an empty reactor were studied under differential conversions (X<10%) to understand the nature of the reaction. The rate dependence on pressure and temperature are shown in FIGS. 1 and 2, respectively. FIG. 1 is a Reaction Order Plot for the Empty Reactor (30 cm³), 450° C., 1 to 18 bar C₂H₄, conversion <10%, and FIG. 2 is an Arrhenius Plot for the Empty Reactor (12 cm³), 1.5 bar C₂H₄, 340-500° C., conversion <2%. The reaction order between 1 and 18 bar was 2.1, indicative of a second order reaction in ethylene concentration. The activation energy over the range of 340-500° C. at atmospheric pressure was 228 KJ/mol. Table 1 shows the ethylene conversion at various temperatures and pressures in an empty reactor. Table 2 shows the product distributions.

TABLE 1
Ethylene Conversion at Various Temperatures
and Pressures in an Empty Reactor.

| Temperature | Pressure | Flow Rate | Conversion |
|---|---|---|---|
| (° C.) | (bara) | (sccm) | (%) |
| 336 | 43.5 | 77 | 0.1 |
| 404 | 43.5 | 68 | 9.7 |
| 410 | 29 | 156 | 21 |
| 410 | 36 | 156 | 31 |
| 430 | 25 | 156 | 29 |
| 430 | 31 | 156 | 44 |
| 430 | 37 | 156 | 61 |
| 432 | 21 | 142 | 3.7 |
| 432 | 21 | 52 | 11 |
| 460 | 1.5 | 6 | 0.4 |
| 460 | 1.5 | 1.4 | 2.1 |
| 465 | 15 | 156 | 21 |
| 465 | 25 | 156 | 56 |
| 465 | 31.5 | 156 | 74 |

TABLE 2
Product Distributions at Different T, P, and
Ethylene Conversions in an Empty Reactor

| | | | | | | | | | |
|---|---|---|---|---|---|---|---|---|---|
| Temperature | (° C.) | 336 | 404 | 430 | 432 | 432 | 460 | 460 | 465 |
| Pressure | (bara) | 43.5 | 43.5 | 37 | 21 | 21 | 1.5 | 1.5 | 31.5 |
| Conversion | (%) | 0.1 | 9.7 | 61 | 3.7 | 11 | 0.4 | 2.1 | 74 |
| CH₄ | mol % | 0.5 | 0.3 | 0.3 | 0.4 | 0.5 | 0.9 | 1.1 | 1.5 |
| C₂H₆ | mol % | 0 | 0.3 | 1.1 | 1.9 | 1 | 1.6 | 0 | 1.8 |
| C₃H₈ | mol % | 0 | 0.3 | 0.3 | 0.5 | 0.5 | 0 | 0 | 2.1 |
| C₃H₆ | mol % | 2.2 | 7 | 12.2 | 13.2 | 15.9 | 73.1 | 73.8 | 26.7 |
| C₄ | mol % | 19.4 | 25.4 | 27.1 | 24.6 | 28.3 | 14.8 | 15 | 29.2 |
| C₅ | mol % | 18.3 | 21.8 | 20.2 | 20.9 | 23.3 | 5 | 5.3 | 17.1 |
| C₆ | mol % | 22.7 | 21.6 | 15.3 | 13.7 | 14.6 | 3.4 | 3.4 | 9.8 |
| C₇ | mol % | 13.8 | 10.8 | 10.7 | 11.4 | 9.8 | 1.4 | 1.1 | 6.5 |
| C₈ | mol % | 19.5 | 9.5 | 7.8 | 8.5 | 4.5 | 0 | 0.4 | 3.6 |
| C₉ | mol % | 3.2 | 2.7 | 4.5 | 4.8 | 1.7 | 0 | 0 | 1.6 |
| C₁₀ | mol % | 0 | 0.2 | 0.6 | 0 | 0 | 0 | 0 | 0.1 |

### Example 2

High purity grade (>99%) amorphous silica (Davisil 636) was purchased from Sigma-Aldrich, having an average pore diameter of 6.0 nm, surface area of 480 m²/g, and pore volume of 0.75 cm³/g. The particle size distribution was 200-500 microns (35-60 mesh). The amorphous silica was loaded into the thermal zone (about 30 cm³) and reacted with flowing ethylene (Table 3). At three different temperature and pressure conditions, the conversion achieved with amorphous SiO₂ occupying the thermal reaction zone was higher than in the empty reactor. At 340° C. and 1.5 bara, the conversion was 15 times higher with SiO₂ than the empty reactor. For comparison, at 380° C. and 1.5 bara, the conversion was 4 times the empty reactor and at 340° C., 43 bar, the conversion was two times higher. These results demonstrate that the addition of SiO₂ to the thermal reaction enhances the rate leading to higher conversions.

TABLE 3
Rate with SiO₂ vs Empty Reactor

| | | | | | Rate |
|---|---|---|---|---|---|
| | Temper- | | Flow | | enhancement |
| | ature | Pressure | Rate | Conversion | vs empty |
| | (° C.) | (bar) | (sccm) | (%) | reactor |
| Empty Reactor | 340 | 1.5 | 6.1 | 6 × 10⁻⁴ | — |
| SiO₂ | 340 | 1.5 | 6.1 | 9 × 10⁻³ | 15x |
| Empty Reactor | 380 | 1.5 | 6.1 | 8 × 10⁻³ | — |
| SiO₂ | 380 | 1.5 | 6.1 | 3 × 10⁻² | 4x |
| Empty Reactor | 340 | 43 | 77 | 0.1 | — |
| SiO₂ | 340 | 43 | 51 | 0.3 | 2x |

Table 4 below summarizes the product distribution when SiO₂ was added to the reactor. The products varied slightly. At 340° C., 43 bar and a conversion of roughly 0.2%, the SiO₂ resulted in twice as much C₄ and C₉ products but less C₅, C₆, and C₇ products as compared to the empty reactor. At 2.6% conversion (380° C., 43 bara), the results with SiO₂ resembled the empty reactor results more closely with roughly the same product selectivity of C₃, C₆, and C₇, but with slightly more C₄ and C₉ produced, and less C₅ and C₈.

TABLE 4

| Products with SiO₂ vs Empty Reactor (mole %) | | | | |
|---|---|---|---|---|
| T (° C.) | 340 | 340 | 380 | 380 |
| P (bara) | 43 | 43 | 43 | 43 |
| X (%) | 0.1 | 0.3 | 2.6 | 2.8 |
| Mole selectivity | Empty | | Empty | |
| (%) | Reactor | SiO₂ | Reactor | SiO₂ |
| CH₄ | 0.5 | 0.2 | 0.1 | 0.2 |
| C₂H₆ | 0.0 | 0.0 | 0.2 | 0.2 |
| C₃H₈ | 0.0 | 0.0 | 0.0 | 0.0 |
| C₃H₆ | 2.2 | 1.6 | 4.5 | 4.2 |
| C₄ | 19.4 | 45.3 | 23.3 | 29 |
| C₅ | 18.3 | 7.4 | 18.2 | 15.6 |
| C₆ | 22.7 | 18.7 | 24.7 | 23.6 |
| C₇ | 13.8 | 7.3 | 11.1 | 10.6 |
| C₈ | 19.5 | 12.2 | 13.5 | 11.7 |
| C₉ | 3.2 | 7.3 | 3.7 | 4.2 |
| C₁₀ | 0.0 | 0.0 | 0.8 | 0.7 |

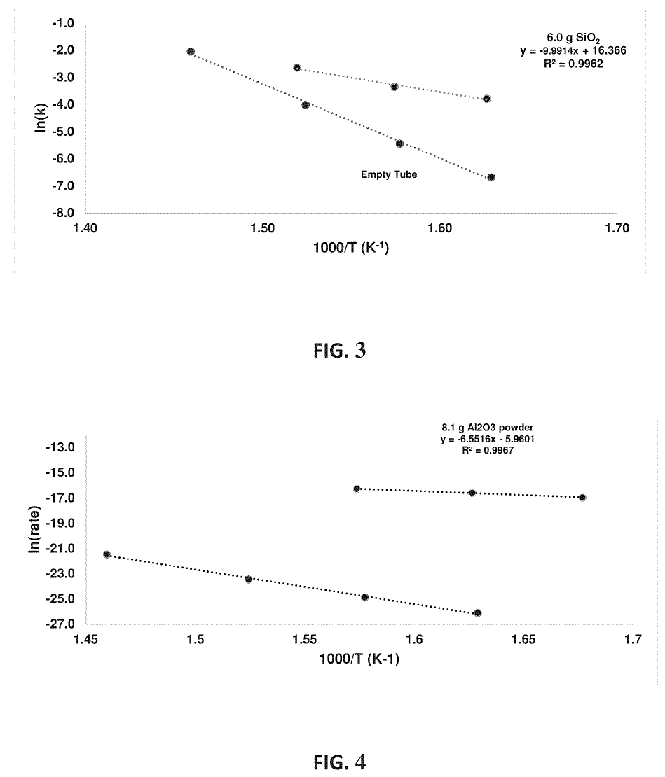

The activation energy from 340-380° C. was also determined with the SiO₂ added to the reactor. FIG. 3 is an Arrhenius Plot for SiO₂, 1.5 bar C₂H₄, 320-360° C., conversion <2% compared to the empty reactor. The empty reactor rate data (lower line) was plotted together with the SiO₂ rate data to demonstrate that the addition of SiO₂ resulted in a lower activation energy of 89 KJ/mol compared to 228 KJ/mol with no SiO₂ added, signifying that the silica support significantly enhanced the reaction rate by lowering the activation barrier for the reaction to take place.

### Example 3

High purity Catalox Sba 200 γ-alumina was obtained from Sasol, having a reported average pore size of 4-10 nm, surface area of 200 m²/g, and pore volume of 0.35-0.5 cm³/g. This alumina is powder form with an average size of 45 microns. To prevent pressure build-up in the bed, the powder was sieved to 25-50 mesh (300-700 microns) and then added to the reactor thermal zone. A substantial rate enhancement was demonstrated with the γ-Al₂O₃ (see Tables 5 and 6). Table 5 demonstrates significant rate enhancement at four different temperatures and pressures. At 340° C., 1.5 bar, an ethylene conversion of 2.0% was achieved with Al₂O₃ compared to 6×10⁻⁴% without Al₂O₃, a rate enhancement of 10,000 times. At 360° C., 1.5 bar, the conversion with Al₂O₃ was 8.4% compared to 2×10⁻³ without Al₂O₃, a factor of 4,200 higher rate. In fact, with Al₂O₃ an ethylene conversion of 35% was obtained at 350° C. and 35 bar by filling the entire thermal zone (about 30 cm³) with Al₂O₃ powder (˜40 g), corresponding to a rate of 1.9×10⁻⁶ mol C₂H₄/s/cm³. In the empty reactor, this rate was only attainable above 400° C. While there was some evidence of coking, the high reaction rate was steady for over a day under these conditions, resulting in the collection of several vials of liquid products.

TABLE 5
Rate with Al₂O₃ vs Empty Reactor

| | | | | | Rate |
|---|---|---|---|---|---|
| | Temper- | | Flow | | enhancement |
| | ature | Pressure | Rate | Conversion | vs empty |
| | (° C.) | (bar) | (sccm) | (%) | reactor |
| Empty Reactor | 340 | 1.5 | 6.1 | 6 × 10⁻⁴ | — |
| Al₂O₃ | 340 | 1.5 | 18.5 | 2.0 | 10,000x |
| Empty Reactor | 360 | 1.5 | 6.1 | 2 × 10⁻³ | — |
| Al₂O₃ | 360 | 1.5 | 6.1 | 8.4 | 4,200x |
| Empty Reactor | 310 | 43 | 77 | 2 × 10⁻² | — |
| Al₂O₃ | 300 | 35 | 117 | 9.0 | 600x |
| Empty Reactor | 350 | 35 | 117 | 3.5 | |
| Al₂O₃ | 350 | 35 | 117 | 35.0 | 10x |

The data in Table 6 demonstrates in more detail the high ethylene conversions that was accomplished with enhancement from the addition of alumina to the reactor volume. In general, the addition of alumina allowed a lower reactor temperature (e.g. 350° C.) to obtain significantly higher conversions (e.g., 89%), which was only achievable at significantly higher temperatures (>450° C.) in the empty reactor. For example, conversions as high as 89% were demonstrated with alumina at 350° C., whereas, without any alumina added, the highest conversion achieved at this temperature was less than 10%. The alumina was also studied at higher temperatures, such as 430° C., resulting in rate enhancement even with partial loading (1.5 g of Al₂O₃, about 10%) of the thermal zone.

TABLE 6
High Ethylene Conversion at Various Temperatures
and Pressures with Al2O3 in the Reactor and for
the one conversion without any alumina (*).

| Temperature | Mass of | Pressure | Flow Rate | Conversion |
|---|---|---|---|---|
| (° C.) | Alumina (g) | (bara) | (sccm) | (%) |
| 350 | 40 | 35 | 100 | 35 |
| 350 | 40 | 35 | 50 | 75 |
| 350 | 40 | 35 | 25 | 89 |
| 350 | 0 | 35 | 25 | <10 |
| 430 | 1.5 | 25 | 117 | 31 |
| 430 | 1.5 | 27 | 78 | 55 |
| 430 | 1.5 | 35 | 78 | 79 |
| 430 | 1.5 | 35 | 92 | 66 |
| 430 | 1.5 | 36 | 117 | 70 |
| 430 | 1.5 | 36 | 155 | 66 |
| 430 | 1.5 | 39 | 92 | 80 |

The product selectivity with the addition of Al₂O₃ also differed from the empty reactor. At 43 bara and 360° C., three times as much propylene and slightly less than twice as many butenes were produced with Al₂O₃ than in the empty reactor. At 1.5 bar, more C₅-C₉ products were made with the addition of Al₂O₃ than in the empty reactor at 360° C. Another key difference was the production of ethane. At 7.2% conversion at 360° C. and 43 bar, the ethane selectivity was 23.1 mole %. At similar conditions, the empty reactor produced 0.7 mole % ethane; however, at 1.5 bar and 360° C., the ethane produced by Al₂O₃ was 10.4 mole %. Table 7 summarizes these results below.

TABLE 7

| Products with Al₂O₃ vs Empty Reactor (mole %) | | | | |
|---|---|---|---|---|
| Temp (° C.) | 360 | 360 | 360 | 360 |
| Pres. (bar) | 43 | 43 | 1.5 | 1.5 |
| X (%) | 0.5 | 7.2 | 1 × 10⁻³ | 7.7 |
| Molar selectivity | Empty | | Empty | |
| (%) | Reactor | Al₂O₃ | Reactor | Al₂O₃ |
| CH₄ | 0.1 | 1.4 | 0.0 | 0.7 |
| C₂H₆ | 0.7 | 23.1 | 0.0 | 10.4 |
| C₃H₈ | 0.0 | 1.2 | 0.0 | 1.9 |
| C₃H₆ | 3 | 10.9 | 61.0 | 36.6 |
| C₄ | 22.1 | 38.8 | 39.0 | 26.9 |
| C₅ | 15.2 | 5.9 | 0.0 | 11.8 |
| C₆ | 25.7 | 9.9 | 0.0 | 5.5 |
| C₇ | 10.5 | 3.4 | 0.0 | 3.1 |
| C₈ | 14.5 | 3.7 | 0.0 | 1.5 |
| C₉ | 7 | 1.3 | 0.0 | 0.6 |
| C₁₀ | 1.2 | 0.1 | 0.0 | 0.0 |

FIG. 4 is an Arrhenius Plot for Al₂O₃, 1.5 bar C₂H₄, 320-360° C. and 410-470° C. compared to the empty reactor. The activation energy of Al₂O₃ powder was determined to be 55 KJ/mol from 320 to 360° C. compared to only 228 KJ/mol in the empty reactor, signifying that the Al₂O₃ support significantly enhanced the thermal radical reaction.

### Example 4: Na-BEA

Ammonium form zeolite Beta (NH₄—[Al]BEA) was purchased from Zeolyst with a Si/Al ratio of 12.5. It was converted to H-form BEA (H—[Al]BEA) by calcining at 500° C. for 3 hr. The Brønsted acid (H⁺) sites were removed by converting H—[Al]BEA to Na-BEA by two successive ion exchanges at 25° C. with 1.0 M NaNO₃ solution in which the pH was gradually adjusted to 8-9 using 0.5 M NaOH and allowed to stir overnight (˜12 hours). The pH was measured to be 8-9 after stirring overnight without any extra NaOH added. The resulting slurry was washed 4-5 times with ultrapure water and centrifuged to remove any excess NaNO₃. It was then dried overnight at 125° C. and calcined the next day at 300 C for 3 hr. In all calcinations, a ramp rate of 1.5° C./min was used. Na-BEA powder was then sieved to 25-50 mesh.

Table 8 shows the results of the non-acidic zeolite, Na-BEA, to enhance the rate of thermal reaction. At 340° C. and 1.5 bar, the ethylene conversion was 1.2% with Na-BEA but only 6×10⁻⁴% in the empty reactor, a factor of 2,100 times more. At 360° C. and 1.5 bar, the rate enhancement was 1,600 times, in which the conversion was 3.0% with Na-BEA and 2×10⁻³% in the empty reactor. At 380° C., the rate enhancement was 700 times, with a conversion of 5.6% with Na-BEA versus 8×10⁻³% in the empty reactor. Table 8 summarizes these results.

TABLE 8
Rate with Na-BEA vs Empty Reactor

| | | | | | Rate |
|---|---|---|---|---|---|
| | Temper- | | Flow | | enhancement |
| | ature | Pressure | Rate | Conversion | vs empty |
| | (° C.) | (bar) | (sccm) | (%) | reactor |
| Empty Reactor | 340 | 1.5 | 6.1 | 6 × 10⁻⁴ | — |
| Na-BEA | 340 | 1.5 | 6.5 | 1.2 | 2,100x |
| Empty Reactor | 360 | 1.5 | 6.1 | 2 × 10⁻³ | — |
| Na-BEA | 360 | 1.5 | 6.5 | 3.0 | 1,600x |
| Empty Reactor | 380 | 1.5 | 6.1 | 8 × 10⁻³ | — |
| Na-BEA | 380 | 1.5 | 6.5 | 5.6 | 700x |

The product distribution with Na-BEA was also different from the empty reactor results (Table 9). At 360° C. and 1.5 bar, the empty reactor case resulted in only C₃H₆ and butenes with no higher molecular weight products detected. In contrast, the addition of Na-BEA resulted in about 24 mol % products in the C₅ and above range, in addition to propylene and butenes as observed in the empty reactor. There were minor amounts of ethane and methane produced (3.4 mol % and 0.5 mol %, respectively) with Na-BEA, which did not occur in the empty reactor. The presence of Na-BEA also resulted in a higher degree of branching in the form of isobutene (about 30% of the C₄ products) which was not observed in the empty reactor ethylene reaction.

TABLE 9

| Products with Na-BEA vs Empty Reactor | | | | |
|---|---|---|---|---|
| Temperature (° C.) | 360 | 360 | 380 | 380 |
| Pressure (bara) | 1.5 | 1.5 | 1.5 | 1.5 |
| X (%) | 1 × 10⁻³ | 3.0 | 8 × 10⁻³ | 5.6 |
| Mole selectivity | Empty | | Empty | |
| (%) | Reactor | Na-BEA | Reactor | Na-BEA |
| CH₄ | 0.0 | 0.5 | 0 | 0.7 |
| C₂H₆ | 0.0 | 3.4 | 0 | 4.2 |
| C₃H₈ | 0.0 | 1.0 | 0 | 1.6 |
| C₃H₆ | 61.0 | 27.5 | 46.1 | 28.3 |
| C₄ | 39.0 | 39.8 | 31.1 | 39.9 |
| C₅ | 0.0 | 11.6 | 19.1 | 15.9 |
| C₆ | 0.0 | 8.5 | 3.7 | 7.7 |
| C₇₊ | 0.0 | 3.4 | 0 | 1.7 |

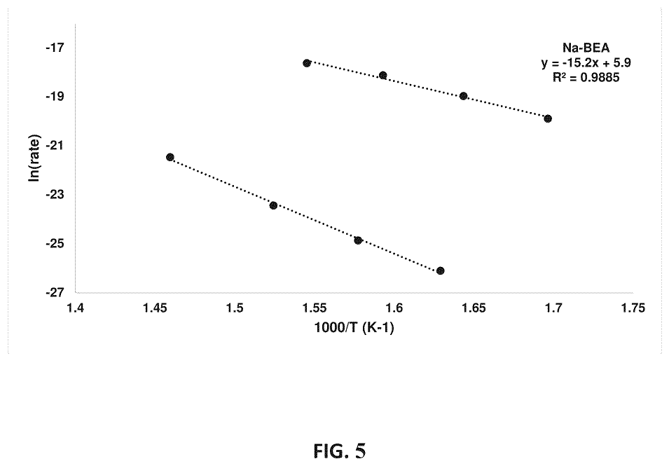

FIG. 5 is an Arrhenius Plot for Na-BEA, 1.5 bar C₂H₄, 320-380° C., conversion <5% compared to the empty reactor. The activation energy of Na-BEA was determined to be 126 KJ/mol from 320° C. to 380° C. compared to 228 KJ/mol in the empty reactor, signifying that the Na-BEA support significantly enhances the thermal radical reaction.

### Example 5

Another non-acidic zeolite, Na—Y, was used to enhance the rate of thermal reaction. Sodium (Na⁺) form of a commercial faujasite zeolite (Na—Y) was obtained with a Si/Al ratio of 2.6 and a surface area of >500 m²/g. To prevent pressure build-up in the bed, the powder was sieved to 25-50 mesh (300-700 microns). Table 10 summarizes the results of using this non-acidic zeolite. At 380° C. and 1.5 bar, the ethylene conversion was 0.4% with Na—Y but only 2×10⁻³% in the empty reactor, a factor of 400 times larger. At 400° C. and 1.5 bar, the rate enhancement was 12 times, in which the conversion was 1.7% with Na—Y and 0.1% in the empty reactor.

TABLE 10
Rate with Na-Y vs Empty Reactor

| | | | | | Rate |
|---|---|---|---|---|---|
| | Temper- | | Flow | Con- | enhancement |
| | ature | Pressure | Rate | version | vs empty |
| | (° C.) | (bar) | (sccm) | (%) | reactor |
| Empty Reactor | 380 | 1.5 | 6.1 | 8 × 10⁻³ | — |
| Na-Y | 380 | 1.5 | 6.0 | 0.4 | 400x |
| Empty Reactor | 410 | 1.5 | 2.8 | 0.1 | — |
| Na-Y | 400 | 1.5 | 3.0 | 1.7 | 12x |

The product distribution with Na—Y was different from the empty reactor results (Table 11). At 380° C. and 1.5 bar, the empty reactor case resulted in only about 23 mol % selectivity to C₅ products and above. In contrast, the addition of Na-BEA resulted in about 36 mol % products in the C₅ and above range, in addition to propylene and butenes as observed in the empty reactor. There were minor amounts of ethane and methane produced (2.0 mol % and 1.0 mol %, respectively) with Na—Y which did not occur in the empty reactor. The presence of Na—Y also resulted in a slightly higher degree of branching in the form of isobutene, which was not observed in the empty reactor ethylene reaction.

TABLE 11

| Products with Na-Y vs Empty Reactor | | |
|---|---|---|
| Temperature (° C.) | 380 | 380 |
| Pressure (bara) | 1.5 | 1.5 |
| X (%) | 1 × 10⁻³ | 0.4 |
| | Empty Reactor | Na-Y |
| CH₄ | 0 | 1.0 |
| C₂H₆ | 0 | 2.0 |
| C₃H₈ | 0 | 1.2 |
| C₃H₆ | 46.1 | 16.0 |
| C₄ | 31.1 | 43.3 |
| C₅ | 19.1 | 15.8 |
| C₆ | 3.7 | 10.2 |
| C₇₊ | 0 | 10.5 |

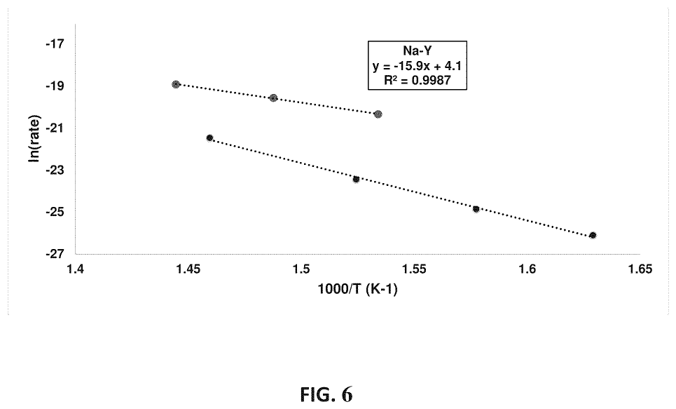

FIG. 6 is an Arrhenius Plot for Na—Y, 1.5 bar C₂H₄, 320-380° C., conversion <5% compared to the empty reactor. The activation energy of Na—Y was also determined to be 132 KJ/mol from 320° C. to 380° C. compared to 228 KJ/mol in the empty reactor, signifying that the Na—Y support significantly enhances the thermal radical reaction.

### Example 6

Additional ethylene conversion rates were measured from 300 to 400° C. at conversions below 5%. In each case, a higher ethylene conversion rate was observed by packing the reactor with high surface area SiO₂ or Al₂O₃, evidenced at about 43 bar, as shown in Table 12. Since the densities were different, each had a different weight loading. Furthermore, the thermal background conversion was subtracted from the rates of the supports. At 340° C. and 43 bar, the gas phase conversion was 0.082% at a gas-hourly space velocity (GHSV) of 8.0 hr-1, whereas with Al₂O₃, the conversion was 5.0% at a GHSV of 81 hr-1. With SiO₂, a GHSV of 5.4 hr-1 resulted in 0.32% ethylene conversion. Thus, the conversion rate was about 600 times higher in the presence of Al₂O₃ and only 2.6 times higher in the presence of SiO₂. The SiO₂ and Al₂O₃ were also compared to each other by normalizing with the surface area (see Table 12). The SiO₂ had the larger surface area per gram (480 m²/g) compared to Al₂O₃ (200 m²/g). However, the Al₂O₃ was about 270 times more reactive than SiO₂ per m² available. Therefore, the available surface area alone does not control the rate of ethylene conversion on these two high surface area materials.

TABLE 12
Comparison of Ethylene Conversion Rates at
340° C. and 42.0-43.5 bar C₂H₄

| | | | Rate per | Rate per |
|---|---|---|---|---|
| | GHSV | Conversion | Surface | Volume |
| | (hr⁻¹) | (%) | Area (vs SiO₂) | (vs Thermal) |
| Thermal, Gas | 8.0 | 0.082 | — | 1 |
| Phase | | | | |
| SiO₂ | 5.4 | 0.32 | 1 | 2.6 |
| Al₂O₃ | 81 | 5.0 | 270 | 620 |

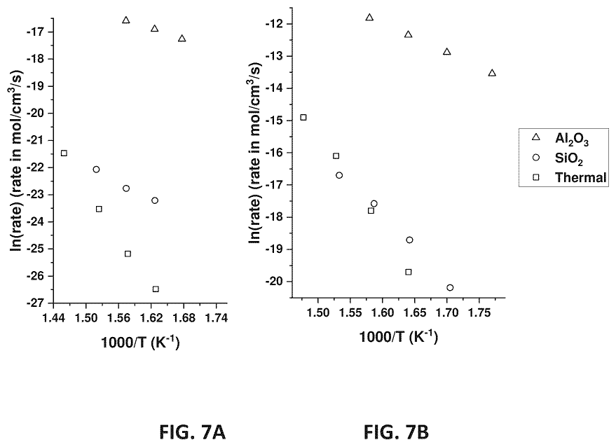

Estimates for the reaction order were made from the rate measurements at 1.5 and 43 bar. At 340° C., the orders for SiO₂ and Al₂O₃ were 1.4 and 1.1, respectively, compared to the gas phase order of 1.9. The observed Arrhenius relationship revealed a lower apparent activation energy for each support compared to the empty reactor, which is about 244 KJ/mol at both 1.5 (FIG. 7A) and 43.5 bar (FIG. 7B). At 1.5 bar, the apparent activation energies for SiO₂ and Al₂O₃ were 89 and 55 KJ/mol, respectively. At 43 bar, these values for SiO₂ and Al₂O₃ were 176 and 76, respectively. Thus, SiO₂ showed a 100% higher activation energy at 43 bar, whereas the Al₂O₃ was 35% higher.

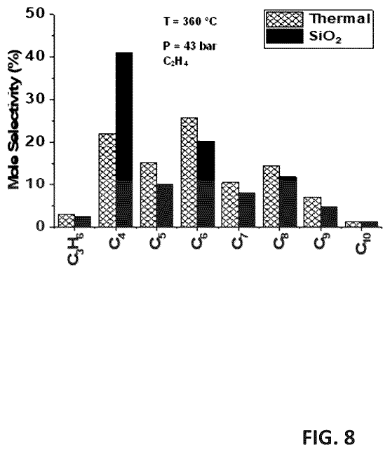

The products for ethylene conversion in the presence of SiO₂ and Al₂O₃ were also compared to the gas phase reaction at high pressure, and the two displayed unique behaviors. SiO₂ was reacted with ethylene at 360° C. and 43 bar, giving a conversion of 0.96%. For comparison, thermally the conversion was 0.58%. The carbon number distributions, shown in FIG. 8, are very similar for SiO₂ and the gas phase reaction, with one noticeable difference, the selectivity to C₄ products were about twice as high with SiO₂. Given that the relative rate of ethylene reaction with SiO₂ is only about twice the thermal rate, the data suggest that the presence of SiO₂ gives rise to more C₄ products. The C₄ linear isomer distributions in Table 13 show that double bond isomerization occurs with SiO₂ compared to gas phase reaction, which produces about 80% 1-butene. With SiO₂, 1-butene is 44% of C₄, with the remainder being mostly trans- and cis-2-butene. The amount of n-butane is very small (ca. 1%) in both cases. Furthermore, there was no evidence of increased branched products such as isobutane or isobutene compared.

TABLE 13
C4 product distribution for ethylene conversion in the presence of SiO₂
compared to the thermal reaction at 360° C., 43.0-43.5 bar.
The conversions thermally and with SiO₂ were 0.58 and 0.96%.

| | C₄ Product Distribution | | | |
|---|---|---|---|---|
| Catalyst | n-Butane | 1-Butene | cis-2-Butene | trans-2-Butene |
| None | — | 80 | 8 | 11 |
| (Thermal) | | | | |
| SiO₂ | — | 44 | 24 | 31 |

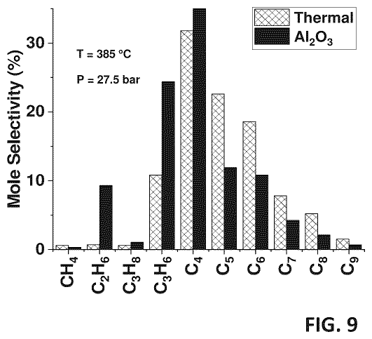

Alumina gave rise to a unique product distribution. The products were compared to the thermal reaction at 385° C. and 27.5 bar around 15% conversion. Since the rate with Al₂O₃ was much higher compared to the thermal rate, the conversions at high pressure with a fully packed reactor of Al₂O₃ were too high to compare. Thus, in one experiment, only about 10% of the reactor was filled (3 g of Al₂O₃). The thermal reaction rate was verified to be less than 10% of the rate with 3 g of Al₂O₃, and the small thermal background conversion was subtracted from the products reported in FIG. 9, which shows the product selectivity of ethylene in the presence of alumina compared to the thermal reaction at 385° C. and 27.5 bar. The conversions thermally and with alumina were 11.9 and 16.9%, respectively. Despite subtracting the thermal background, the carbon number distributions share many features, such as little methane production (<2%) and the presence of many non-oligomer products, resulting in analogous apparent single-carbon growth patterns. For example, the most abundant products with Al₂O₃ at these conditions is C₄ followed by propylene (35 and 25%, respectively). The remaining higher MW products (C₅₊) decrease in order of increasing carbon number. The same is true for the thermal reaction, except that there is less propylene and more C₅ to C₉ products. Thermally, C₄ was the most abundant carbon number group (nearly 32%) while propylene was just 11%.

The two major differences observed here between Al₂O₃ and the gas phase reaction were the production of ethane and the C₄ distribution. With Al₂O₃, there is about 9% selectivity to ethane, whereas in the thermal reaction ethane is less than 1% of the products. The C₄ distribution, shown in Table 14, features the introduction of branched C₄ products, which were not observed thermally. Isobutene and isobutane were collectively 18% of the C₄, and 1-butene was only 14% of C₄. The remaining C₄ were 2-butenes (63%). The gas phase reaction produced 1-butene with 71% selectivity among C₄ isomers.

TABLE 14
C4 product distribution of ethylene conversion in the presence of alumina
compared to the thermal reaction at 385° C., 27.5 bar. The conversions
thermally and with alumina were 11.9 and 16.9%, respectively.

| | % C₄ Product Distribution | | | | | |
|---|---|---|---|---|---|---|
| | | | | | cis-2- | trans-2- |
| Catalyst | n-Butane | iso-Butane | iso-Butene | 1-Butene | Butene | Butene |
| None | 3 | — | — | 71 | 10 | 16 |
| (Thermal) | | | | | | |
| Al₂O₃ | — | 3 | 15 | 14 | 26 | 37 |

In summary, the comparative study of SiO₂ and Al₂O₃ demonstrated varying levels of ethylene rates compared to the thermal reaction, as well as product alterations. Al₂O₃ provided the best yield to desired products C₃+ and introduced branched C₄ products. Therefore, Al₂O₃ was studied over a wider range of conditions to determine the effects of temperature, pressure, and conversion on the products with ethylene.

### Example 7: Further Studies of the Reaction of Ethylene in the Presence of Al₂O₃ (Alumina)

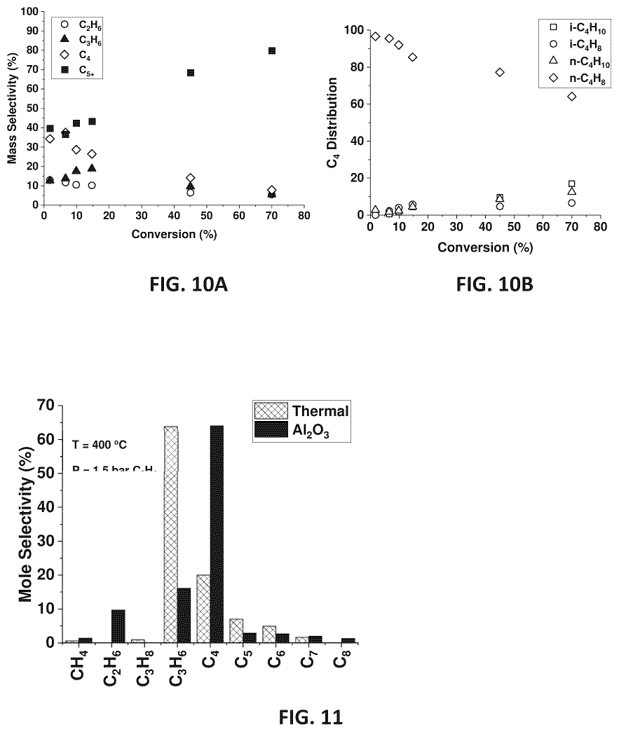

Product distribution at higher ethylene conversions with Al₂O₃ were first determined. At 360° C. and 23 bar, by varying the space velocity, ethylene conversions from 1 to 70% were obtained. At the same conditions, the purely thermal reaction was not significant. The higher activity with alumina is consistent with the measured kinetic rates at lower conversions since, as mentioned earlier, comparing the rates with alumina at high temperature and pressure resulted in about two orders of magnitude higher rate than thermally. The molecular-weight distributions as a function of conversion in FIGS. 10A-10B demonstrate the high selectivity to C₅₊ products. FIG. 10A shows the major products selectivity and FIG. 10B shows the C₄ isomer distribution. During this experiment, a light-yellow liquid product was condensed into an in-line vial submerged in an ice bath. The weight-hourly space velocity (WHSV) required to obtain 70% conversion was 0.1 hr⁻¹. Nonetheless, the high conversion remained stable for at least 24 hours. Approximately 1 cm³ of liquid hydrocarbons were condensed per hour during the 24 hour duration of the trial. At the end of the experiment, the Al₂O₃ in the center of the packed bed displayed a yellow-orange color, but was not black, suggesting coking was not significant at this temperature. In our previous study of the thermal reactions of ethylene, conversions near 70% required temperatures greater than 450° C., and the resulting carbon number distribution produced more light olefins such as propylene and 1-butene, and less C₅₊.

FIGS. 10A and 10B illustrate several noteworthy observations about the reaction pathway, with the major products being C₅+, C₄, propylene, and ethane. Methane was less than about 1% selective at each conversion. The ethane selectivity decreased steadily with increasing conversion, starting as high as 15% at 2% conversion, but becoming only about 5% at 70% conversion. Conversely, the C₅₊ production increased over the same conversion range from about 40 to 80% of the products. The propylene and C₄ selectivity profiles appear to increase in the early stages of the reaction (below 20% conversion), however decrease steadily after reaching maximum selectivity values, signifying their importance in secondary reactions leading to C₅₊ liquids.

As was seen earlier in FIG. 9, the comparison of the product distribution with the gas phase reaction at similar conversion shared some product behavior, namely the presence of non-oligomer products. Since secondary reactions become important at higher conversions, i.e. above 1%, an experiment was conducted to compare the reaction with alumina compared to the gas phase at the early stage of the reaction (X<1%).

FIG. 11 shows the product selectivity of ethylene with alumina vs the thermal reaction at 1.5 bar, 400° C. and 0.1% conversion. FIG. 11 shows that at 0.1% conversion at 400° C. and 1.5 bar there is a completely different molecular-weight distribution due to Al₂O₃, despite also sharing several similarities with the gas phase reaction. To compare Al₂O₃ to the thermal reaction at 0.1% conversion, 0.25 g of Al₂O₃ were loaded. The thermal conversion at the same flow rate was at least 50 times lower so that the products observed were only due to Al₂O₃. In a separate experiment, employing a much lower flow rate. 0.1% conversion was achieved thermally for comparison. With Al₂O₃, the major products were 60% C₄ followed by 15% C₃H₆ and 10% ethane, in stark contrast to the gas phase which produces about 65% propylene. 20% C₄, and little ethane. Both the thermal reaction and Al₂O₃ produce less than 1.5% methane at this temperature. Along with propylene, non-oligomer products such as C₅ and C₇ were present in comparable amounts to oligomers such as C₆ and C₈.

The C₄ isomer distribution further highlights the influence Al₂O₃ has on the reaction, Table 15. The C₄ products were 68% 2-butenes and only 25% 1-butene, whereas for the gas phase reaction 1-butene was about 69%. While the amount of isobutene produced is small (4%), it is nonetheless relatively more than produced without Al₂O₃ (<0.1%). Butadiene is also produced to a small extent in both cases (near 2%). No butane was observed with Al₂O₃, although about 3% selectivity was seen thermally.

TABLE 15
C4 product distribution of ethylene conversion in the presence of alumina compared
to the thermal reaction at 1.5 bar C₂H₄, 400° C., and 0.1% conversion.

| | % C₄ Product Distribution | | | | | |
|---|---|---|---|---|---|---|
| | | | | | cis-2- | trans-2- |
| Catalyst | n-Butane | Butadiene | iso-Butene | 1-Butene | Butene | Butene |
| None | 3 | 2 | — | 69 | 10 | 15 |
| (Thermal) | | | | | | |
| Al₂O₃ | — | 2 | 4 | 25 | 28 | 40 |

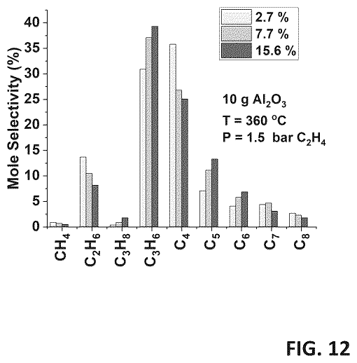

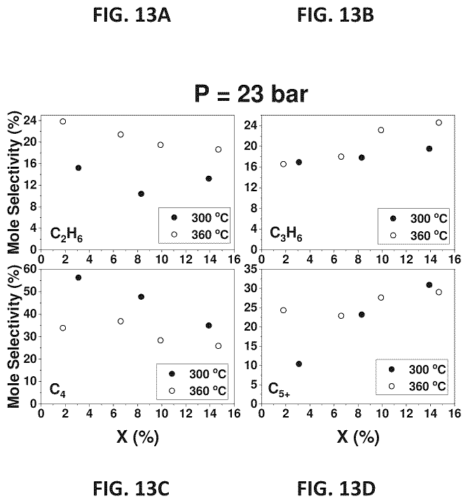

The products were next studied to identify the effects of increasing conversion below 20%, with an emphasis on tracking changes in the molecular-weight and isomer distributions. FIG. 12 shows the product selectivity of ethylene with alumina versus conversion below 20% at 1.5 bar and 360° C. FIGS. 13A-13D show the selectivity of major products with alumina at 300 vs 360° C. at 23 bar of C₂H₄ below 20% conversion. FIG. 13A shows the shows selectivity of ethane. FIG. 13B shows the shows product selectivity of propane. FIG. 13C shows the shows product selectivity of butane. FIG. 13D shows the shows product selectivity of pentane and heavier. At 1.5 bar and 360° C. the conversion was varied from 2.7 to 15.6% (FIG. 12). The molecular weight distribution, in FIGS. 13A-D, demonstrated several clear trends. Importantly, C4 decreased from about 35 to 25% whereas C5 increased from 7 to 13% and C6 from 3 to 7%. The propylene selectivity increased from 31 to about 40%. The ethane selectivity decreased from 14 to 8%. However, the propane selectivity increased from <0.5 to 2%.

The C4 isomer behavior offer several notable observations, Table 16. The C4 distribution, which is mostly 2-butenes (50-70%), demonstrated a notable increase in branched isomers, with isobutene and isobutane increasing from 15% to 24% combined of C4. Additionally, the ratio of isobutene to isobutane decreased from >50 at 2.7% conversion to 2.1 at 15.6% due to increasing isobutane production.

TABLE 16
C4 product distribution of ethylene conversion in the presence of
alumina compared at 1.5 bar C₂H₄, 400° C., and 0.1% conversion.

| | % C₄ Product Distribution | | | | | |
|---|---|---|---|---|---|---|
| % | | | | | cis-2- | trans-2- |
| Conversion | n-Butane | iso-Butane | iso-Butene | 1-Butene | Butene | Butene |
| 2.7 | — | — | 15 | 17 | 27 | 40 |
| 7.7 | — | 5 | 22 | 15 | 23 | 34 |
| 15.6 | 1 | 11 | 23 | 10 | 21 | 34 |

The reactions of ethylene with Al2O3 reveal a transition in product behavior above about 400° C., shifting from higher MW products below 400° C. to decomposition products (methane, ethane, coke) above 400° C. The selectivity to methane increased from less than 1% at 300 C to 14% at 470° C. Likewise, the ethane selectivity increased from 8 to 47%. The remaining products are mostly propylene and C4 with less than 3% each of C5 to C7. The high selectivity to saturated products were accompanied by blackening of the Al2O3 at 470° C., indicating coke deposition. At 300° C., the Al2O3 remained white during the experiment.

For temperatures below 400° C., such as 300° C. compared to 360° C. at 23 bar, at higher temperature more ethane and less C4 was formed at each conversion (see FIGS. 13A-D). The propylene and C5+ showed more complex trends. For example, the propylene selectivity appears the same at both temperatures until about 8% conversion; above 8% conversion more propylene forms at 360° C. Likewise, the C5+ selectivity is nearly the same at both temperatures above 8% conversion but is clearly higher at 360° C. below 8% conversion. As seen in FIGS. 10A-B above, as conversion increases, secondary reactions become important leading to changes in the product distributions. Thus, these features as seen in FIG. 13 highlight the complicated nature of interpreting product changes at different temperatures due to consecutive reactions in oligomerization chemistry.

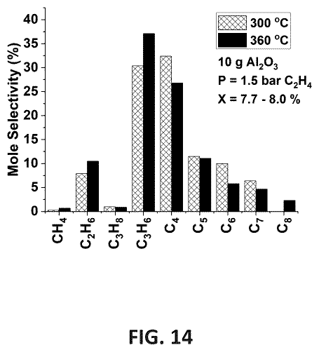

Comparison of the MW and isomer distributions between 30° and 360° C. at 1.5 bar and a single conversion (˜ 8%), seen in FIG. 14, reveals similar features of higher ethane selectivity and lower C₄ selectivity at 360 compared to 300° C. FIG. 14 shows the product selectivity of ethylene with alumina versus temperatures of 300° C. and 360° C. at 7.7-8.0% conversion and 1.5 bar. The 60° C. increase in temperature lead to about twice as much isobutene formed (22 vs 11% of C₄), see Table 17. Thus, there are clear temperature effects on the products, both to the molecular-weight and amount of branched C₄.

TABLE 17
C4 product distribution of ethylene conversion in the presence of
alumina compared at 1.5 bar C₂H₄, 400° C., and 7.7-8.0% conversion.

| | % C₄ Product Distribution | | | | | |
|---|---|---|---|---|---|---|
| Temperature, | | | | | cis-2- | trans-2- |
| ° C. | n-Butane | iso-Butane | iso-Butene | 1-Butene | Butene | Butene |
| 300 | 1 | 5 | 11 | 14 | 26 | 42 |
| 360 | 1 | 5 | 22 | 15 | 23 | 34 |

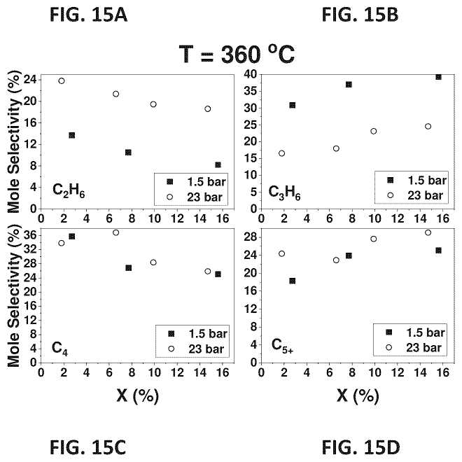

The effects of pressure on the products at 1.5 vs. 23 bar at 360° C. below 20% conversion is depicted in FIGS. 15A-15D. FIG. 15A shows the shows selectivity of ethane. FIG. 15B shows the shows product selectivity of propane. FIG. 15C shows the shows product selectivity of butane. FIG. 15D shows the shows product selectivity of pentane and heavier. Propylene, C4, C5+, and ethane are the main products. Ethane and propylene show the most straightforward pressure dependences. At 360° C., the ethane selectivity at 23 bar is slightly less than twice the selectivity at 1.5 bar. On the contrary, about twice as much propylene is produced at 1.5 bar than 23 bar. Both the C4 and C5+ products appear in comparable amounts at both pressures. Analogous to the effects of temperature in FIG. 13, the products may show complex selectivity profiles due to the competing consecutive reactions.

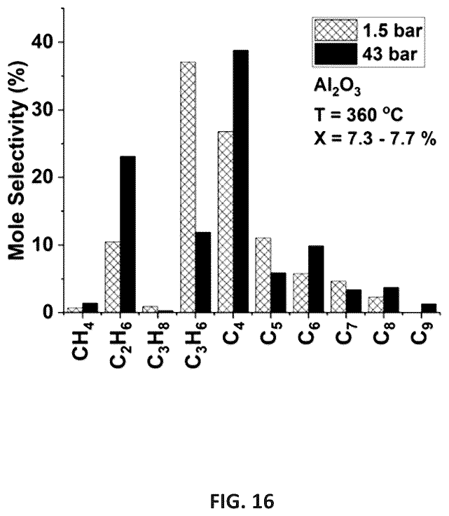

Additionally, at 43 bar and about 8% conversion, most of the same trends are evident, with propylene being 38% at 1.5 bar but only 12% at 43 bar, as shown in FIG. 16. FIG. 16 shows the product selectivity of ethylene with alumina at 1.5 vs 43.0 bar and 360° C. at 7.3-7.7% conversion. The C₄ was 27% at 1.5 bar but comprised 40% of the products at 43 bar. Similarly, there were almost twice as many C₆ products at 43 bar. Thus, a striking observation is that the higher-pressure product distributions apparently highlight earlier stages of the reaction where oligomerization is the main reaction (C₄, C₆, C₈, etc), before cracking results in significant non-oligomers. That is, the even carbon oligomers (C₄, C₆, and C₈) are more abundant than C₃, C₅, C₇, and C₉ non-oligomers, as opposed to the single-carbon growth pattern characteristic at higher reaction extents and lower pressures.

When comparing the effects of conversion, temperature, and pressure on the products, it was apparent that secondary reactions play an important role in the product distributions, especially as conversion increased beyond 20%. Propylene, for example, increased to a maximum selectivity below 20% conversion in FIG. 10A, and then steadily decreased up to 70% conversion. It is therefore insightful to evaluate the reactions of propylene with Al₂O₃, to understand the difference in kinetics and product behavior to ethylene.

### Example 8: Reaction of Propylene in the Presence of Al₂O₃

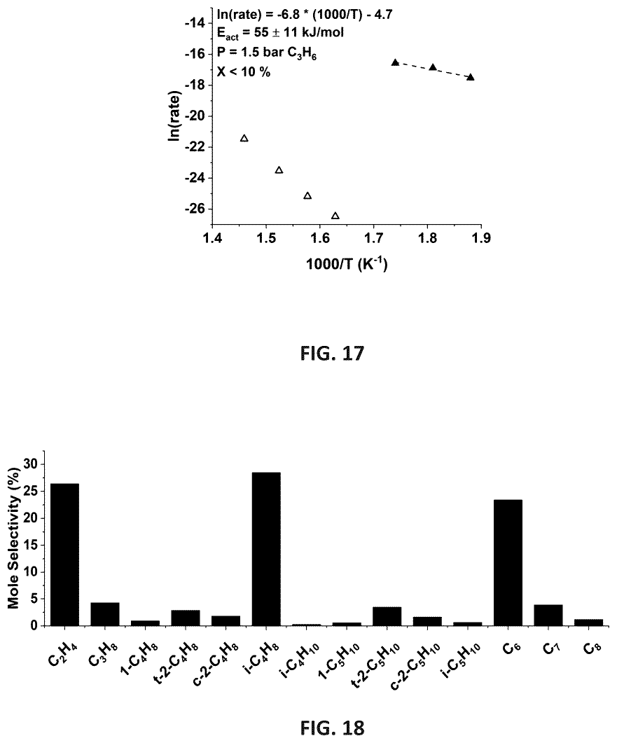

The Arrhenius parameters for propylene with Al₂O₃ were measured from 260 to 300 C at 1.5 bar, as shown in FIG. 17. FIG. 17 is an Arrhenius plot depicting the kinetics of propylene reactions with alumina from 260 to 300° C. at 1.5 bar. X<10%. The thermal reactions of propylene from 400 to 500° C. are shown in open triangles for comparison. Over this temperature range, the apparent activation energy was measured to be 55 KJ/mol, the same value as ethylene. The conversion of C3H6 at 300 C and 1.5 bar was 7.4% at a GHSV of 61 br-1. For comparison, at the same conditions, the C2H4 conversion was 0.85% at a GHSV of 20 hr-1. Thus, propylene reacts about an order of magnitude faster than ethylene at 300 C and 1.5 bar, an important distinction since propylene is one of the main products early in the reaction (below 20% conversion) and could result in more facile bond activation leading to increased ethylene consumption or higher MW products.

When pure propylene was reacted over Al₂O₃ from 260 to 300 C at 1.5 bar, the molecular weight distribution revealed a disproportionation bad occurred, with ethylene and isobutene as the major products, forming in a 1:1 molar ratio at low conversion (2.0% at 260° C.). FIG. 18 shows the product selectivity of propylene with alumina at 2.0% conversion at 260° C. and 1.5 bar. The product distribution at 2.0% conversion at 260° C. shows that ethylene and isobutene are the main products, comprising 27 and 28% of the products, respectively, followed by C6 (23%). Propane was about 4%, and neither methane nor ethane were detected. Small amounts of n-butenes, C5, C7, and C8 products were also formed (<5% each). The C5 isomers, in contrast to the C4, were mostly linear. Of the C5, only about 10% were isopentenes, and 81% were 2-pentenes.

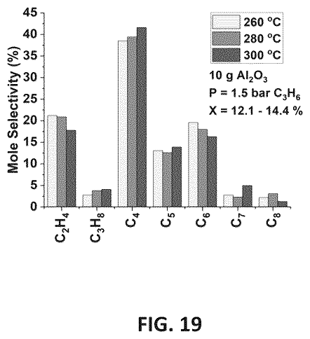

FIG. 19 shows the product selectivity of propylene with alumina versus temperatures from 260 to 300° C. at 12.1-14.4% conversion at 1.5 bar. The effects of increasing temperature from 260 to 300° C. portray several notable shifts. Less ethylene and C6 and more C4 are formed at 300° C. compared to at 260° C. The C4 product distribution, Table 18, reveals a gradual increase in n-butenes compared to isobutene at higher temperatures. Isobutene decreased from 78 to 73% of C4 while n-butenes increased from 16 to 20% at 300° C. compared to 260° C. The C5 experienced a sharper change, with isopentenes increasing from 15% of C5 at 260° C. to 31% at 300° C.

TABLE 18
C4 product distribution with alumina from 260 to 300° C.
at 12.1-14.4% conversion at 1.5 bar from C3H6.

| Temper- | % C₄ Product Distribution | | | | |
|---|---|---|---|---|---|
| ature, | iso- | | | cis-2- | trans-2- |
| ° C. | Butane | iso-Butene | 1-Butene | Butene | Butene |
| 260 | 4 | 78 | 1 | 6 | 10 |
| 280 | 3 | 77 | 2 | 7 | 10 |
| 300 | 3 | 73 | 4 | 8 | 12 |

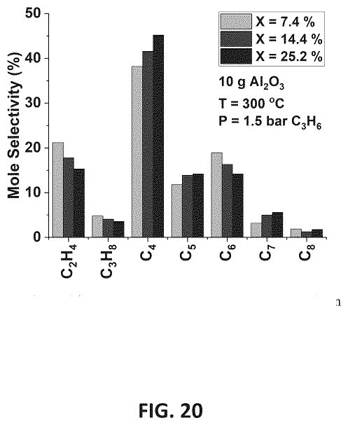

FIG. 20 shows the product selectivity of ethylene with alumina versus temperatures of 300° C. and 360° C. at 7.7-8.0% conversion and 1.5 bar. Increasing the conversion from 7.4 to 25.2% at 300° C. demonstrated clear shifts in selectivity, with ethylene and C6 each decreasing from about 20 to 15 mole % while C4, C5, and C7 increased. Ethylene and C6 both decreased from around 20 to 15 mole. The net increase in C4 appears to be largely due to an increase in isobutane and n-butenes as opposed to the major product, isobutene. Table 19. The isobutene to isobutane ratio decreased with increasing conversion, from 45 to 13.

TABLE 19
C4 product distribution with alumina from 7.4 to 25.2 % conversion at
300C and 1.5 bar from C3H6.

| | % C₄ Product Distribution | | | | |
|---|---|---|---|---|---|
| % | | | | cis-2- | trans-2- |
| Conversion | iso-Butane | iso-Butene | 1-Butene | Butene | Butene |
| 7.4 | 1 | 77 | 4 | 7 | 11 |
| 14.4 | 3 | 73 | 4 | 8 | 12 |
| 25.2 | 5 | 67 | 4 | 9 | 14 |

### Specific Embodiments

Specific embodiments of the present disclosure can further include any one or more of the following numbered paragraphs:

1. A method for making light hydrocarbon oligomers, comprising reacting one or more C₂ to C₁₂ olefins with at least one porous support material at a temperature of about 200° C. to 500° C. to provide a higher molecular weight product comprising C₃ to C₂₆ olefins, wherein the at least one porous support material contains no additional catalytic metals, activators, promoters or acid sites.
2. The method of paragraph 1, wherein the porous support material is Al₂O₃, SiO₂, or a non-acidic zeolite.
3. The method of paragraph 1 or 2, wherein the porous support material is Na-BEA or Na-Y.
4. The method of any paragraph 1 to 3, wherein the porous support material has a pore size of about 5 Å to about 500 Å, and a surface area of about 25 m²/g to about 600 m²/g.
5. The method of any paragraph 1 to 5, wherein the one or more C₂ to C₁₂ olefins are reacted at a pressure of about 1 Bar (g) to about 100 Bar (g).
6. The method of any paragraph 1 to 5, wherein the one or more C₂ to C₁₂ olefins consist essentially of ethylene and propylene.
7. The method of any paragraph 1 to 6, wherein the higher molecular weight product consists essentially of C₄ to C₂₆ olefins.
8. The method of any paragraph 1 to 7, wherein the higher molecular weight product consists essentially of C₁₂ to C₂₀ olefins having a boiling point in the range of 170° C. to 360° C.
9. A method for making light hydrocarbon oligomers, consisting of reacting one or more C₂ to C₁₂ olefins with at least one porous support material at a temperature of about 200° C. to 500° C. to provide a higher molecular weight product comprising C₃ to C₂₆ olefins, wherein the at least one porous support material contains no additional catalytic metals, activators, promoters or acid sites.
10. The method of paragraph 9, wherein the porous support material is Al₂O₃, SiO₂, or a non-acidic zeolite.
11. The method of paragraph 9 or 10, wherein the porous support material is Na-BEA or Na—Y.
12. The method of any paragraph 9 to 11, wherein the porous support material has a pore size of about 5 Å to about 500 Å, and a surface area of about 25 m²/g to about 600 m²/g.
13. The method of any paragraph 9 to 12, wherein the one or more C₂ to C₁₂ olefins are reacted at a pressure of about 1 Bar (g) to about 100 Bar (g).
14. The method of any paragraph 9 to 13, wherein the one or more C₂ to C₁₂ olefins consist essentially of ethylene and propylene.
15. The method of any paragraph 9 to 14, wherein the higher molecular weight product consists essentially of C₄ to C₂₆ olefins.
16. The method of any paragraph 9 to 15, wherein the higher molecular weight product consists essentially of C₁₂ to C₂₀ olefins having a boiling point in the range of 170° C. to 360° C.
17. A light hydrocarbon oligomer made from a method, comprising reacting one or more C₂ to C₁₂ olefins with at least one porous support material at a temperature of about 200° C. to 500° C. to provide a higher molecular weight product comprising C₃ to C₂₆ olefins, wherein the at least one porous support material contains no additional catalytic metals, activators, promoters or acid sites, wherein the higher molecular weight product is a mixture of olefins having odd and even carbon numbers.
18. The oligomer of paragraph 17, wherein the one or more C₂ to C₁₂ olefins consist essentially of ethylene and propylene.
19. The oligomer of paragraph 17, wherein the one or more C₂ to C₁₂ olefins consist essentially of ethylene and the higher molecular weight product comprises C₃ and C₅ olefins.
20. The oligomer of paragraph 17, wherein the one or more C₂ to C₁₂ olefins consist essentially of ethylene and the higher molecular weight product comprises at least 2 wt % C₃ olefins and at least 2 wt % C₅ olefins.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, meaning the values take into account experimental error, machine tolerances and other variations that would be expected by a person having ordinary skill in the art.

The foregoing has also outlined features of several embodiments so that those skilled in the art can better understand the present disclosure. Those skilled in the art should appreciate that they can readily use the present disclosure as a basis for designing or modifying other methods or devices for carrying out the same purposes and/or achieving the same advantages of the embodiments disclosed herein. Those skilled in the art should also realize that such equivalent constructions do not depart from the spirit and scope of the present disclosure, and that they can make various changes, substitutions, and alterations herein without departing from the spirit and scope of the present disclosure, and the scope thereof is determined by the claims that follow.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

1. A method for making light hydrocarbon oligomers, comprising:
reacting one or more C2 to C12 olefins with at least one porous support material at a temperature of about 200° C. to 500° C. to provide a higher molecular weight product comprising C3 to C26 olefins, wherein the at least one porous support material contains no additional catalytic metals, activators, promoters or acid sites.

2. The method of claim 1, wherein the porous support material is Al₂O₃, SiO₂, or a non-acidic zeolite.

3. The method of claim 1, wherein the porous support material is Na-BEA or Na—Y.

4. The method of claim 2, wherein the porous support material has a pore size of about 5 Å to about 500 Å and a surface area of about 25 m2/g to about 600 m2/g.

5. The method of claim 1, wherein the one or more C2 to C12 olefins are reacted at a pressure of about 1 Bar (g) to about 100 Bar (g).

6. The method of claim 5, wherein the one or more C2 to C12 olefins consist essentially of ethylene and propylene.

7. The method of claim 1, wherein the higher molecular weight product consists essentially of C4 to C26 olefins.

8. The method of claim 1, wherein the higher molecular weight product consists essentially of C12 to C20 olefins having a boiling point in the range of 170° C. to 360° C.

9. A method for making light hydrocarbon oligomers, consisting of:
reacting one or more C2 to C12 olefins with at least one porous support material at a temperature of about 200° C. to 500° C. to provide a higher molecular weight product comprising C3 to C26 olefins, wherein the at least one porous support material contains no additional catalytic metals, activators, promoters or acid sites.

10. The method of claim 9, wherein the porous support material is Al₂O₃, SiO₂, or a non-acidic zeolite.

11. The method of claim 9, wherein the non-acidic zeolite is Na-BEA or Na—Y.

12. The method of claim 10, wherein the porous support material has a pore size of about 5 Å to about 500 Å and a surface area of about 25 m2/g to about 600 m2/g.

13. The method of claim 9, wherein the one or more C2 to C12 olefins are reacted at a pressure of about 1 Bar (g) to about 100 Bar (g).

14. The method of claim 13, wherein the one or more C2 to C12 olefins consist essentially of ethylene and propylene.

15. The method of claim 9, wherein the higher molecular weight product consists essentially of C4 to C26 olefins.

16. The method of claim 9, wherein the higher molecular weight product consists essentially of C12 to C20 olefins having a boiling point in the range of 170° C. to 360° C.

17. A method for making a light hydrocarbon oligomer comprising:
reacting one or more C2 to C12 olefins with at least one porous support material at a temperature of about 200° C. to 500° C. to provide a higher molecular weight product comprising C3 to C26 olefins, wherein the at least one porous support material contains no additional catalytic metals, activators, promoters or acid sites, wherein the higher molecular weight product is a mixture of olefins having odd and even carbon numbers.

18. The oligomer of claim 17, wherein the one or more C2 to C12 olefins consist essentially of ethylene and propylene.

19. The oligomer of claim 17, wherein the one or more C2 to C12 olefins consist essentially of ethylene and the higher molecular weight product comprises C3 and C5 olefins.

20. The oligomer of claim 17, wherein the one or more C2 to C12 olefins consist essentially of ethylene and the higher molecular weight product comprises at least 2 wt % C3 olefins and at least 2 wt % C5 olefins.
